(19) United States
(12) Patent Application Publication
(10) Pub. No.: US 2025/0367431 A1
(43) Pub. Date: Dec. 4, 2025

(54) SYSTEMS AND METHODS FOR DETERMINING A TOTAL BLOOD VOLUME FLOW IN A CARDIAC SUPPORT SYSTEM AND VASCULAR SUPPORT SYSTEM

(71) Applicant: KARDION GMBH, Stuttgart (DE)

(72) Inventors: Hardy Baumbach, Stuttgart (DE); Karin Schneider, Herrenberg (DE); Inga Schellenberg, Stuttgart (DE); Martina Budde, Karlsruhe (DE); Thomas Alexander Schlebusch, Renningen (DE)

(21) Appl. No.: 19/232,575

(22) Filed: Jun. 9, 2025

Related U.S. Application Data
(63) Continuation of application No. 15/734,010, filed on Jul. 9, 2021, now Pat. No. 12,324,906.

(51) Int. Cl. A61M 60/523; A61M 60/17; A61M 60/178; A61M 60/216; A61M 60/411

(52) U.S. Cl. CPC A61M 60/523; A61M 60/17; A61M 60/178; A61M 60/216; A61M 60/411; A61M 2205/3327; A61M 2205/3368; A61M 2205/3666; A61M 2205/70; A61M 2230/50

(57) ABSTRACT

The invention relates to a method for determining a total fluid volume flow (1) in the region of an implanted vascular support system (2), comprising the following steps: a) determining a reference temperature (3) of the fluid, b) determining a motor temperature (4) of an electric motor (5) of the support system (2), c) determining the thermal dissipation loss (6) of the electric motor (5), d) ascertaining the total fluid volume flow (1) using the reference temperature (3), the motor temperature (4), and the thermal dissipation loss (6) of the electric motor (5).

### INCORPORATION BY REFERENCE TO ANY PRIORITY APPLICATIONS

The present application is a continuation of U.S. application Ser. No. 15/734,010 filed on Jul. 9, 2021, which is a National Phase entry of PCT Patent Application No. PCT/EP2019/064802, filed on Jun. 6, 2019, titled METHOD FOR DETERMINING A TOTAL FLUID VOLUME FLOW IN THE REGION OF AN IMPLANTED VASCULAR SUPPORT SYSTEM AND IMPLANTABLE VASCULAR SUPPORT SYSTEM, which is an International Application of and claims the benefit of priority to German Patent Application No. 102018208879.9, filed on Jun. 6, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention relates to a method for determining a total fluid volume flow in the region of an implanted vascular support system, a processing unit, and an implantable vascular support system. The invention is in particular used in (fully) implanted left-heart support systems (LVAD).

### Description of the Related Art

Implanted left-heart support systems (LVAD) mainly exist in two design variants. On the one hand, there are (percutaneous) minimally invasive left-heart support systems. The second variant are left-heart support systems invasively implanted under the chest opening. The variant according to the first variant conveys blood directly from the left ventricle into the aorta since the (percutaneous) minimally invasive left-heart support system is positioned centrally in the aortic valve. The second variant conveys the blood from the left ventricle via a bypass tube into the aorta.

The task of a cardiac support system is to convey blood. In this respect, the so-called heart-time volume (HTV, usually indicated in liters per minute) is highly clinically relevant. In other words, the heart-time volume in this case relates to the total volume flow of blood (from a ventricle), in particular from the left ventricle, to the aorta. Correspondingly clear is the attempt to collect this parameter as a measured value during operation of a cardiac support system.

Depending on the level of support, which describes the proportion of the volume flow conveyed by a conveying means, such as a pump of the support system, to the total volume flow of blood from the ventricle to the aorta, a certain volume flow reaches the aorta via the physiological path through the aortic valve. The heart-time volume or the total volume flow (Q_{HTV}) from the ventricle to the aorta is therefore usually the sum of the pump volume flow (Qₚ) and the aortic valve volume flow (Qₐ).

An established method for determining the heart-time volume (Q_{HTV}) in the clinical setting is the use of dilution methods, which, however, all rely on a transcutaneously inserted catheter and therefore can only provide heart-time volume measurement data during cardiac surgery. An established method for measuring the pump volume flow (Qₚ) is the correlation of the operating parameters of the support system, predominantly the electrical power consumption, possibly supplemented by further physiological parameters, such as the blood pressure. The integration of dedicated ultrasound measurement technology into a support system has also already been proposed.

A (fully) implanted detection of the heart-time volume, i.e., of Q_{HTV}, in particular by the support system itself, has not yet been proposed or realized. Fully implanted means, in particular, that the means required for the detection are completely located in the body of the patient and remain there. This makes it possible to detect the heart-time volume even outside of cardiac surgery.

### SUMMARY

The object of the invention is to specify an improved method for determining a total fluid volume flow in the region of an implanted vascular support system and to create an improved implantable vascular support system.

In particular, it is an object of the invention to specify a method for determining a total fluid volume flow in the region of an implanted vascular support system and to create an implantable vascular support system by means of which a total fluid volume flow in a blood flow region can be determined in a human or animal body, in which the vascular support system is implanted or arranged.

This object is achieved by the method specified herein and the implantable vascular support system specified herein.

A method for determining a total fluid volume flow in the region of an implanted vascular support system comprises the following steps:
a) determining a reference temperature of the fluid,
b) determining a motor temperature of an electric motor of the support system,
c) determining the thermal dissipation loss of the electric motor,
d) ascertaining the total fluid volume flow using the reference temperature, the motor temperature, and the thermal dissipation loss of the electric motor.

The vascular support system is preferably a cardiac support system, particularly preferably a ventricular support system. The “total volume flow” in particular refers to the total volume flow through a blood vessel or through a cross section of the blood vessel. The blood vessel is, for example, the aorta, in particular in the case of a left-heart support system, or the common trunk (Truncus pulmonalis) into the two pulmonary arteries, in particular in the case of a right-heart support system, preferably the aorta. The method preferably serves to determine a total fluid volume flow from a ventricle of a heart, in particular from a (left) ventricle of a heart, to the aorta in the region of a (fully) implanted, (left) ventricular (heart) support system. The fluid is regularly blood. The support system is preferably arranged at the exit of the left ventricle of the heart or the left heart chamber. The support system is particularly preferably arranged in the aortic valve position.

The method is in particular suitable for determining the total heart-time volume (HTV, formula symbol Q_{HTV}) of a patient, in particular with (fully) implanted left ventricular heart support system (LVAD) in the aortic valve position and/or by the support system itself. The method is based in particular on (thermally) anemometric (measuring) principles for flow measurement. The basic principle in this case is that a flowing medium cools a hot body as a function of the flow speed. The method advantageously allows the heart-time volume to also be made available outside of the surgical scenario with comparable quality as when using a dilution catheter. This is particularly advantageous since the heart-time volume (Q_{HTV}) has a greater clinical relevance than the pump volume flow (Qₚ), which is mostly used and only quantifies the flow through the support system itself.

A particular advantage of the method is that, unlike as usual in anemometric methods, no separate heating element is required to generate the heat flow to be measured. Rather, the thermal dissipation loss, which in any case occurs on the electric motor of the LVAD, can be used for anemometric flow measurement. Preferably, no (separate) heating element (except the electric motor) is used to determine the total fluid volume flow. In other words, the electric motor is the only heating element that is used in the solution proposed here. In particular, in the solution proposed here, the thermal dissipation loss occurring on and/or in the electric motor of the support system is used for the (thermally) anemometric or calorimetric flow measurement. It is furthermore preferred that the support system has no (separate) heating element (except the electric motor).

A reference temperature of the fluid is determined, in particular measured, in step a). The reference temperature is preferably determined by a reference temperature sensor, which is particularly preferably a component of the support system. The reference temperature sensor can, for example, be arranged in and/or on an (inlet) cannula of the support system. The reference temperature usually represents a background temperature of the fluid, in other words a fluid temperature which is in particular not influenced by the thermal dissipation loss of the electric motor.

In step b), a motor temperature of an electric motor of the support system is determined, in particular measured. The electric motor can be a component of a flow machine or of a pump of the support system. The support system is preferably arranged on or in the fluid flow such that a heat flow from the support system, in particular from its electric motor, can be dissipated to the fluid flow. The term “motor temperature” can also be understood to mean an internal temperature or (external) surface temperature of the support system, in particular in the region of the electric motor, which in particular allows a preferably direct conclusion about the temperature of the electric motor, in particular about the temperature of a coil package of the electric motor.

The support system is preferably implanted such that it is located in the fluid flow at least partially, preferably completely, or with at least 50%, particularly preferably at least 85%, or even at least 95% of its (external) surface. Furthermore, the support system is preferably located along at least 50%, particularly preferably at least 85%, or even at least 95% of its length in the fluid flow. One end of the support system in the region of which or on which the electric motor is located is preferably at least partially located in the aorta. Furthermore, the opposite end of the support system in the region of which or on which a (n) (inlet) cannula of the support system is located is preferably located at least partially in a ventricle (the left ventricle) of the heart. Furthermore, the support system is preferably arranged in a blood vessel, such as an artery, in particular the aorta, at least partially, preferably completely, or with at least 50%, particularly preferably at least 85%, or even at least 95% of its (external) surface. The support system is particularly preferably implanted such that it is (completely) located in the (descending) aorta.

In step c), the thermal dissipation loss of the electric motor is determined. The thermal dissipation loss of the electric motor is preferably determined by a current sensor, which preferably measures an electrical current of the electric motor.

In step d), the total fluid volume flow is determined using the reference temperature, the motor temperature, and the thermal dissipation loss of the electric motor. In step d), with the aid of at least one heat transfer specification, at least one heat transfer coefficient, at least one calibration factor, and/or at least one blood vessel cross section, in particular an aortic cross section, the total fluid volume flow is determined as a function of the reference temperature, the motor temperature, and the thermal dissipation loss of the electric motor.

According to an advantageous embodiment, it is proposed that the reference temperature is measured in particular spatially and/or temporally before heating the fluid by the electric motor. A reference temperature sensor is preferably arranged at a distance from the electric motor, in particular upstream of the electric motor, preferably on a (n) (inlet) cannula of the support system. The reference temperature sensor is particularly preferably arranged in the region of and/or on an end of the (inlet) cannula opposite the electric motor.

According to an advantageous embodiment, it is proposed that the motor temperature of the electric motor is measured on a surface along which the fluid flows. The surface is generally an (external) surface of the support system that is in contact with the fluid. The motor temperature can, for example, be measured with a motor temperature sensor which is arranged on an (external) surface of the support system in the region of the (internal) electric motor. Alternatively, the motor temperature of the electric motor can be measured inside the motor. For this purpose, a motor temperature sensor can be arranged inside the electric motor.

According to an advantageous embodiment, it is proposed that a flow speed of the fluid is determined, in particular calculated, in step d) as a function of calibration data, the reference temperature, the motor temperature, and the thermal dissipation loss of the electric motor. The calibration data preferably comprise a characteristic length (e.g., tube diameter, possibly approximated in the region of the aortic valve), a kinematic viscosity of the fluid, a temperature conductivity of the fluid, a thermal conductivity of the fluid, and/or a (top) surface of the support system wetted with fluid.

According to an advantageous embodiment, it is proposed that an ascertained cross-sectional geometry of an aorta in the region of the implanted vascular support system is furthermore taken into account in step d). A (flow) cross section of the aorta in the region of the support system is preferably taken into account. This value can be ascertained by a doctor by means of ultrasound or computer tomography, for example. The total fluid volume flow or the heart-time volume can be particularly advantageously determined, in particular calculated, as a function of the flow speed of the fluid, the (flow) cross section of the aorta and a (speed-dependent) calibration factor. The (speed-dependent) calibration factor can, for example, be ascertained by means of a calibration in the context of implantation, e.g., by using a dilution catheter as the reference standard.

According to an advantageous embodiment, it is proposed that a fluid volume flow which flows through the support system is furthermore determined. In other words, this relates in particular to a fluid volume flow that only flows through the support system itself. This fluid volume flow is usually the so-called pump volume flow (Qₚ), which only quantifies the flow through the support system itself. If this value is known in addition to the total volume flow or heart-time volume (Q_{HTV}), the so-called level of support can be calculated from the ratio of Qₚ to Q_{HTV} (i.e., Qₚ/Q_{HTV}). In order to determine the pump volume flow, an established method for measuring the pump volume flow discussed in the beginning in connection with the prior art can be used.

The total fluid volume flow ascertained in step d) is preferably provided as a control parameter for the support system in a step e), for example. A processing unit of the support system can provide this control parameter as an output variable, in particular to a control unit of the support system that preferably regulates the power of the electric motor and thus in particular also the (blood) delivery rate of the support system.

According to a further aspect, a processing unit is proposed, configured to carry out a method proposed here and comprising a memory in which calibration data are stored. As an alternative or in addition to the calibration data, at least one (speed-dependent) calibration factor and/or a thermal model of the electric motor can also be stored in the memory. In addition, the processing unit can comprise a microprocessor which can access the memory. The processing unit preferably receives data from a reference temperature sensor, a motor temperature sensor, and/or a current sensor.

According to a further aspect, an implantable, vascular support system is proposed, comprising:
a reference temperature sensor for determining a reference temperature of a fluid,
an electric motor,
a motor temperature sensor for determining a motor temperature of the electric motor,
a current sensor for determining at least the current flow through the electric motor or the thermal dissipation loss of the electric motor.

The support system is preferably a left ventricular heart support system (LVAD) or a percutaneous, minimally invasive left-heart support system. Furthermore, the support system is preferably fully implantable. In other words, this means in particular that the means required for the detection, in particular the reference temperature sensor, the motor temperature sensor, and the current sensor, are completely located in the body of the patient and remain there. The support system is particularly preferably configured and/or suitable for being arranged at least partially in a ventricle, preferably in the left ventricle, of a heart and/or in an aorta, in particular in the aortic valve position.

The current sensor is used to determine the current flow through the electric motor and/or the thermal dissipation loss of the electric motor. The current sensor preferably measures the current flow through the electric motor and calculates the dissipation loss of the electric motor therefrom. If the current sensor only supplies the current flow as an output variable, it is in particular provided that the current flow is converted into the dissipation loss of the electric motor in a processing unit of the support system.

The support system furthermore preferably comprises a cannula, in particular an inlet cannula, and a flow machine, such as a pump. The electric motor is regularly a component of the flow machine. The electric motor then drives the flow machine for conveying the fluid. The (inlet) cannula is preferably configured such that in the implanted state, it can guide fluid from a (left) ventricle of a heart to the flow machine. The fluid can be guided through the cannula to the flow machine. The cannula is preferably designed to guide fluid in the form of blood from a (left) ventricle of a heart into an aorta.

The support system is preferably elongated and/or tubular. The inlet cannula and the flow machine are preferably arranged in the region of opposite ends of the support system.

The reference temperature sensor can be arranged on the cannula or near a region of the cannula at a distance from the flow machine. In particular, the reference temperature sensor can be arranged on the cannula or near a region of the cannula facing away from the electric motor. The reference temperature sensor is particularly preferably arranged at a distal end of the cannula, i.e., where the blood flows from a ventricle into the cannula.

The support system can have a tubular elongated structure with a cannula section in which the cannula is formed and with a motor housing section which is connected to the cannula section and in which the electric motor is arranged in a motor housing.

It is advantageous if the reference temperature sensor is arranged in a region of the cannula section at a distance from the motor housing section. The electric motor is preferably arranged in a motor housing around which blood can flow in the aorta.

The support system can furthermore comprise a processing unit configured to determine a total fluid volume flow in the region of the support system using the reference temperature, the motor temperature, and the thermal dissipation loss of the electric motor. The support system is preferably configured to carry out a method proposed here.

The details, features, and advantageous embodiments discussed in connection with the method can also arise accordingly in the processing unit and/or the support system presented here and vice versa. In this respect, reference is made in full to the explanations there regarding the detailed characterization of the features.

### BRIEF DESCRIPTION OF THE DRAWINGS

The solution presented here as well as its technical environment are explained in more detail below with reference to the figures. It should be pointed out that the invention is not to be limited by the exemplary embodiments shown. In particular, unless explicitly stated otherwise, it is also possible to extract partial aspects of the facts explained in the figures and to combine them with other components and/or insights from other figures and/or the present description. The following are shown schematically:

FIG. 1a a percutaneous, minimally invasive left-heart support system,

FIG. 1b a left-heart support system invasively implanted under the chest opening,

FIG. 2 an implanted vascular support system,

FIG. 3 an arrangement of an implanted vascular support system,

FIG. 4 a component architecture of a support system,

FIG. 5 an illustration of a heat flow,

FIG. 6 an illustration of a temperature curve, and

FIG. 7 a further illustration of a temperature curve.

### DETAILED DESCRIPTION

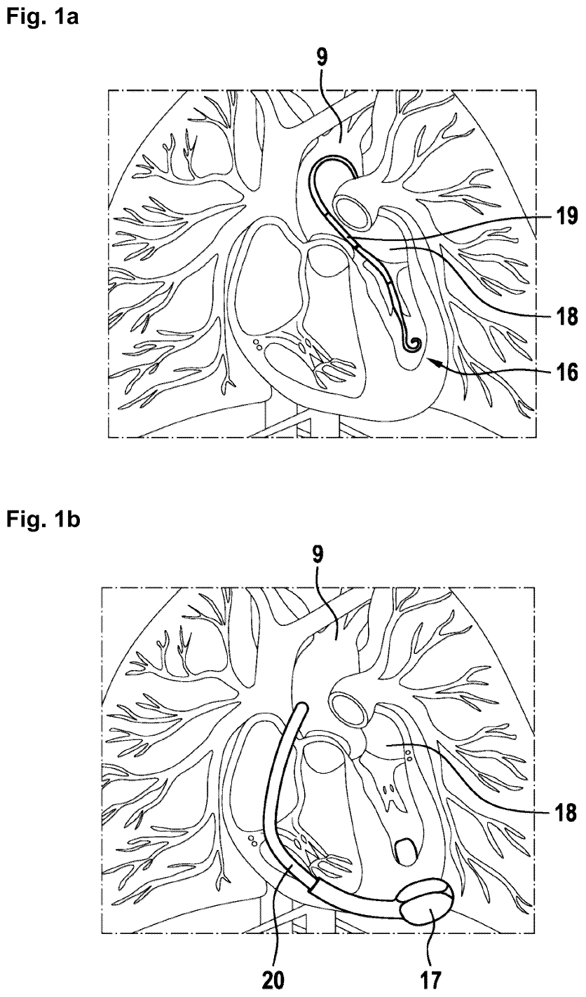

Implanted left-heart support systems (LVAD) exist mainly in two design variants, as shown in FIGS. 1a and 1b. FIG. 1a shows a (percutaneous) minimally invasive left-heart support system 16, while FIG. 1b shows a left-heart support system 17 invasively implanted under the chest opening. The variant according to FIG. 1a conveys blood directly from the left ventricle 18 into the aorta 9 since the (percutaneous) minimally invasive left-heart support system 16 is positioned centrally in the aortic valve 19. The variant according to FIG. 1b conveys the blood from the left ventricle 18 via a bypass tube 20 into the aorta 9.

Depending on the level of support, which describes the proportion of volume flow conveyed by a conveying means, such as a pump of the support system, to the total volume flow of blood from the ventricle 18 to the aorta 9, a certain volume flow reaches the aorta 9 via the physiological path through the aortic valve 19. The heart-time volume or the total volume flow (Q_{HTV}) from the ventricle 18 to the aorta 9 is therefore usually the sum of the pump volume flow (Qₚ) and the aortic valve volume flow (Qₐ).

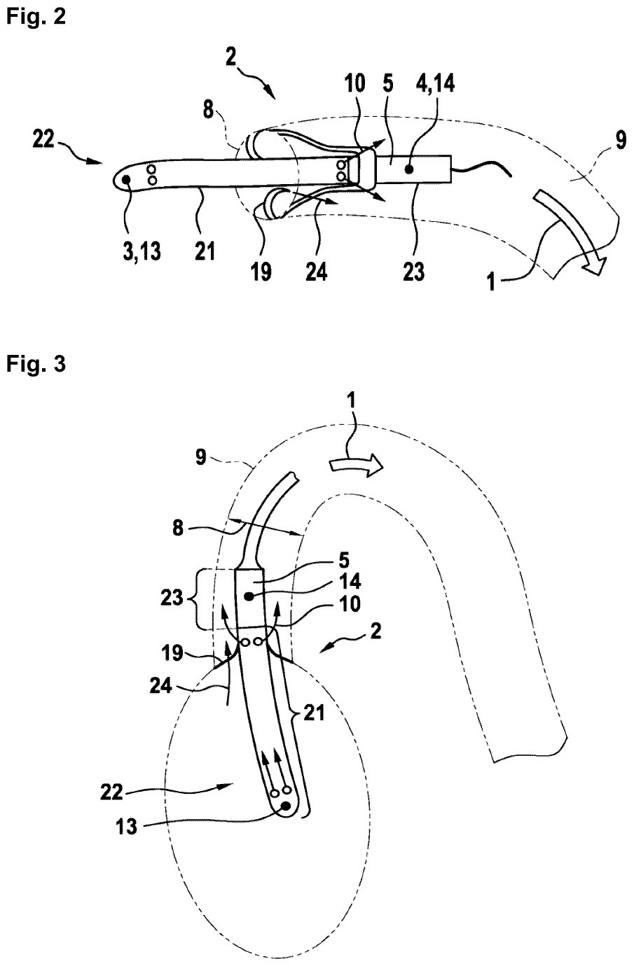

FIG. 2 schematically shows an implantable vascular support system 2 in the aortic valve position. For further illustration, reference is also made simultaneously to the schematic arrangement of the support system 2 according to FIG. 3, wherein the reference signs are used uniformly in all figures.

The support system 2 is here, by way of example, a left ventricular heart support system (LVAD).

The support system has a tubular elongated structure with a cannula section in which an inlet cannula 21 is formed as cannula, and comprises a motor housing section which is connected to the cannula section and in which an electric motor 5 is located in a motor housing 23.

The support system 2 protrudes from the aorta 9 through the aortic valves 19 distally into the ventricle 18. Here, the support system 2 has, by way of example, an inlet cannula 21 which protrudes into the ventricle 18. A fluid volume flow 10 is conveyed, e.g., pumped, through the inlet cannula 21 from the ventricle 18 into the aorta 9 using an electric motor 5 of the support system 2, which drives a flow machine in the form of a pump in the support system 2. Therefore, the fluid volume flow 10 is also referred to as the pump volume flow (Qₚ), which only quantifies the flow through the support system 2 itself.

In addition, it can be seen in FIG. 2 and FIG. 3 that a certain aortic valve volume flow 24 reaches the aorta 9 via the physiological path through the aortic valve 19. The heart-time volume or the total fluid volume flow 1 (Q_{HTV}), passing through a cross-sectional geometry 8 of the aorta 9 in the region of the support system 2, from the ventricle 18 to the aorta 9 is therefore the sum of the fluid volume flow 10 (Qₚ) and the aortic valve volume flow 24 (Qₐ). This is described by the following equation (1).

$${Q}_{HTV} = {Q}_{p} + {Q}_{a} \qquad ( 1 )$$

The support system 2 comprises a reference temperature sensor 13 for determining a reference temperature 3 of a fluid, in this case blood by way of example. The support system 2 furthermore comprises an electric motor 5 and a motor temperature sensor 14 for determining a motor temperature 4 of the electric motor 5. In addition, the support system 2 has a current sensor (not shown here) for determining the thermal dissipation loss (not shown here) of the electric motor 5.

The motor temperature sensor 14 is, by way of example, integrated in a motor housing 23, in which the thermal dissipation loss of the electric motor 5 is dissipated to the surrounding fluid. The motor temperature sensor 14 is configured and arranged such that it can measure the motor temperature 4. For this purpose, the motor temperature sensor 14 can be configured and arranged such that it measures a surface temperature of the motor housing 23 or a temperature of the stator (not shown here) of the electric motor 5. In this case, the temperature of the stator can be approximated by an internal temperature in the motor housing 23 between the motor housing 23 and the coil package (not shown here). Alternatively, the temperature in the coil package can also be measured directly.

The reference temperature sensor 13 detects the reference temperature 3, which here is the background blood temperature by way of example. For this purpose, the reference temperature sensor 13 is positioned in the thermally uninfluenced blood flow upstream of the electric motor 5 representing the heat source; here, by way of example, in the region upstream of the electric motor 5. For this purpose, the reference temperature sensor 13, as shown in FIG. 2, is arranged in a region of the cannula section at a distance from the motor housing section at a distal end of the inlet cannula 21, i.e., where the blood flows from a ventricle into the inlet cannula 21.

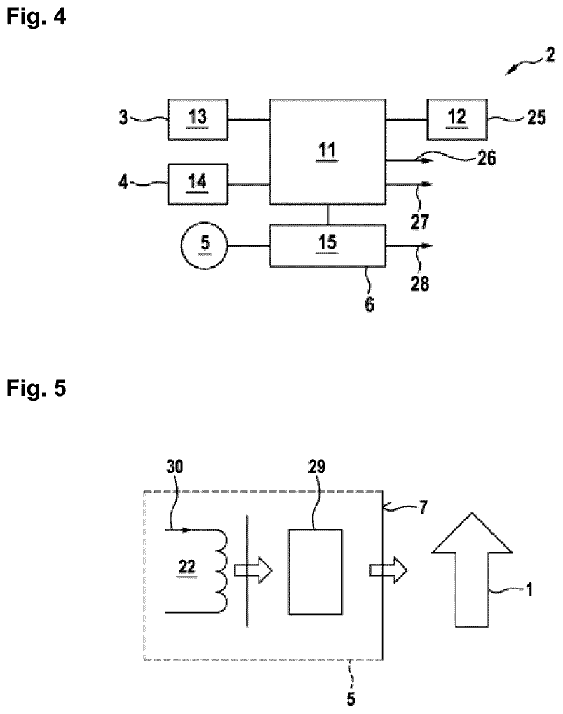

FIG. 4 schematically shows a component architecture of a support system 2. The support system 2 comprises a reference temperature sensor 13 for determining a reference temperature 3 of a fluid, in this case blood by way of example. The support system 2 furthermore comprises an electric motor 5 and a motor temperature sensor 14 for determining a motor temperature 4 of the electric motor 5. In addition, the support system 2 has a current sensor 15 for determining the thermal dissipation loss 6 of the electric motor 5. For this purpose, the current sensor 15 ascertains, by way of example, the current flow (not shown here) through the motor 5 and converts it into the thermal dissipation loss 6. According to the illustration according to FIG. 4, the support system 2 furthermore comprises a processing unit 11 configured to determine a total fluid volume flow (not shown here) in the region of the support system 2 using the reference temperature 3, the motor temperature 4, and the thermal dissipation loss 6 of the electric motor 5. In addition, the support system 2 has an electronically readable memory 12 with calibration data 25.

The measurement data of the reference temperature sensor 13, the motor temperature sensor 14, and the current sensor 15 are transmitted to the processing unit 11. The processing unit 11 processes the measurement data with calibration data 25 from the memory 12 to form the blood flow speed or the (total) blood volume flow. The processing unit 11 furthermore comprises an output 26 to a communication unit (not shown here), an output 27 to a power supply (not shown here), and an output 28 to a motor control (not shown here).

FIG. 5 schematically shows an illustration of an exemplary heat flow (horizontal arrows) through the electric motor 5 to the fluid flow (vertical arrow) or the total fluid volume flow 1. The electric motor 5 in this case comprises, by way of example, a movably mounted rotor (not shown here) and a stationary coil package 22 which is offset by an air gap outside and which is connected to the stator 29. FIG. 5 thus schematically illustrates in other words the thermal conduction transitions from the coil package 22 of the electric motor 5 via the stator 29 to the blood flow. The loss mechanisms in the electric motor 5 primarily relate to the Joule current heat losses Pv (see equation (2) below).

$${P}_{V} = {R}_{TW} \cdot {I}^{2} \qquad ( 2 )$$

Here, R_{TW} denotes the winding resistance of the coil package 22 at the operating temperature T_{W}. The winding resistance R_{TW} in the case of copper is a linear function of the winding temperature T_{W}. This is described by equation (3) below:

$${R}_{TW} = {R}_{25} \cdot ( 1 + {α}_{Cu} ( {T}_{W} - 25 ) ) \qquad ( 3 )$$

with the winding resistance R₂₅ at 25° C., the winding operating temperature T_{W}, and the constant α_{cu}=0.0039K⁻¹.

In addition, iron losses also occur, e.g., magnetization losses according to the following equation (4):

$${P}_{V , magn} = π / 30 \cdot {M}_{Magn} \cdot n \qquad ( 4 )$$

and eddy current losses in the back iron material of the stator according to the following equation (5):

$${P}_{V , Eddy} = const \cdot {n}^{2} \qquad ( 5 )$$

with the number of revolutions n of the motor and the magnetic friction torque M_{Magn}. In addition, bearing losses from the bearing of the motor occur, which are generally negligible.

The thermal resistance between a heat source and a heat sink is measured in Kelvin per watt (K/W). The determining thermal conduction mechanism between the coil package and the blood flow is thermal conduction through the layers of the motor to the outside, as shown in FIG. 5. In order to determine the temperatures, the heat capacities of the individual components traversed by the heat flow as well as the respective heat transfer resistances are required. Since it can be adequately assumed that the electric motor is in stationary operation and thus in thermal equilibrium, the heat capacities are negligible. All necessary parameters can be determined in advance and can be stored in a processing unit.

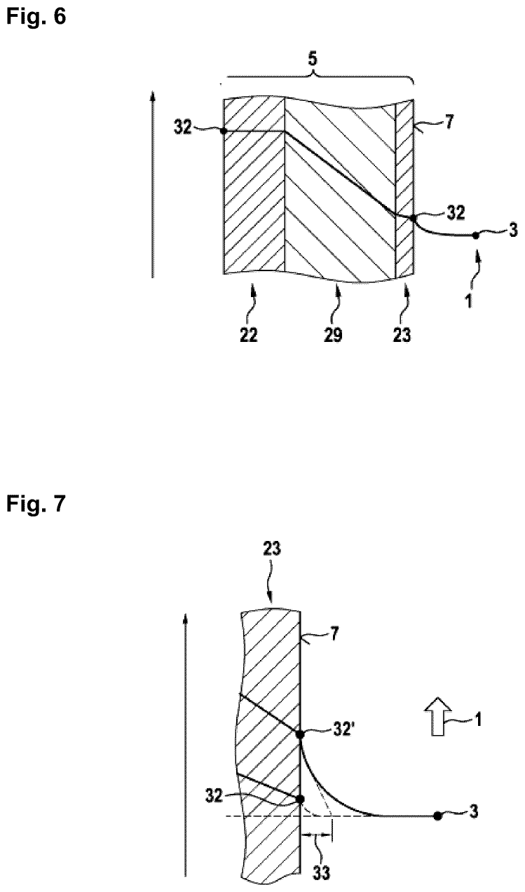

FIG. 6 schematically shows an illustration of a temperature curve along the material layer sequence from the coil package 22 via the stator 29 and the motor housing 23 to the total fluid volume flow 1. FIG. 6 shows a temperature distribution resulting in the thermal equilibrium for a heat flow according to FIG. 5. The highest temperature is present in the heat source, the coil package 22 through which the electrical current flows. The winding temperature 31 (formula symbol T_{W}) of the coil package 22 is therefore the highest temperature in FIG. 6. For simplification, a constant heat distribution over the entire thickness of the coil package 22 was assumed here. Due to the finite thermal conductivity of the stator material and housing material, a linear temperature gradient results via the stator 29 and the motor housing 23, or a logarithmic temperature gradient in the non-simplified case of a cylindrical motor housing 23.

When considering the simplified principle, the winding temperature 31 arising in the coil package 23 (formula symbol T_{W}) is:

$${T}_{W} = {T}_{A} + ( {R}_{th 1} + {R}_{th 2} ) \cdot {P}_{v} \qquad ( 6 )$$

$${T}_{W} = {T}_{A} + ( {R}_{th 1} + {R}_{th 2} ) \cdot {R}_{TW} \cdot {I}^{2} \qquad ( 7 )$$

$${T}_{W} = {T}_{A} + ( {R}_{th 1} + {R}_{th 2} ) \cdot {R}_{25} \cdot ( 1 + {α}_{Cu} ( {T}_{W} - 25 °C . ) ) \qquad ( 8 )$$

$${T}_{W} = {T}_{A} + \frac{( {R}_{th 1} + {R}_{th 2} ) \cdot {R}_{25} \cdot {I}^{2}}{1 - {α}_{Cu} \cdot ( {R}_{th 1} + {R}_{th 2} ) \cdot {R}_{25} \cdot {I}^{2}} \qquad ( 9 )$$

Here, the electrical current flow 30 (formula symbol I) and the surface temperature 32 (formula symbol T_{A}) are the only variable parameters. Rₜₕ₁ describes the thermal resistance between the coil package 22 and the stator 29. Rₜₕ₂ describes the thermal resistance between the stator 29 and the fluid flow. The current flow 30 (formula symbol I) can be ascertained by measuring with the current sensor 15, for example, in a control device of the current sensor, and is thus precisely known. The surface temperature 32 (formula symbol T_{A}) denotes the temperature on a surface 7 of the electric motor 5 along which the fluid flows. In other words, the surface 7 is in the blood stream.

FIG. 7 schematically shows a further illustration of a temperature curve. FIG. 7 shows a detailed view of the illustration according to FIG. 6 in the region of the surface 7 at two different flow speeds. In other words, FIG. 7 illustrates in printed form the dependence of the temperature(s) (surface temperature and thus also stator temperature and thus also coil package temperature) on the flow speed of the fluid flow or of the blood.

As shown in FIG. 7, a liquid film of thickness 33 is formed near the surface 7. The thickness 33 of the liquid film and the temperature difference T_{A}−T_{B} between the surface temperature 32 (formula symbol T_{A}) and the reference temperature 3 (formula symbol T_{B}), which represents the background temperature of the fluid (blood), is a function of the flow speed of the fluid, as illustrated in FIG. 7. According to the illustration in FIG. 7, a lower flow speed of the fluid along the surface 7 leads to a higher surface temperature 32′ than the surface temperature 32, which arises at a comparatively higher flow speed.

The heat flow through the liquid film is

$$\dot{Q} = {α}_{B} ( {T}_{B} - {T}_{A} ) A \qquad ( 10 )$$

with the heat transfer coefficient α_{B} from the top of the housing to the blood and the wetted surface A of surface 7. The heat transfer coefficient is defined as

$${α}_{B} = \frac{Nu λ}{L} \qquad ( 11 )$$

with the dimensionless Nusselt number Nu, the thermal conductivity λ of the fluid (here: blood), and a reference length L, which can be a tube diameter, for example. It furthermore applies to the Nusselt number averaged across the body surface that it is a function of the dimensionless Reynolds number Re and Prandtl number Pr:

$$Nu = f ( Re , Pr ) \qquad ( 12 )$$

These can each be calculated as a function of the geometry and the flow (Re and Pr) or as a function of the fluid properties (Pr) and stored in the calibration data memory. The Reynolds number is defined as

$$Re = \frac{uL}{v} \qquad ( 13 )$$

with the characteristic length L (e.g., tube diameter), the kinematic viscosity of the fluid v, and the sought flow speed u. The Prandtl number is a pure substance variable and given by

$$Pr = \frac{v}{a} \qquad ( 14 )$$

with the temperature conductivity a of the fluid. If the definitions are inserted into the convective heat flow through the liquid film (equation (10)), the relationship between the known heat flow Q and the sought flow speed u is obtained. The result of this insertion is shown in equation (15) below. The heat flow Q is known from an energy balance. It follows from the energy balance for the stationary case considered here that the heat flow Q (in terms of magnitude) substantially corresponds to the thermal dissipation loss 6 (formula symbol Pv).

The surface temperature 32 (formula symbol T_{A}) can be measured here, for example, directly on the surface 7 by means of the motor temperature sensor 14, or the motor temperature sensor 14 can measure a temperature inside the motor and the surface temperature 32 (formula symbol T_{A}) is ascertained from the logarithmic temperature relationship to the temperature distribution in the motor housing (cf. FIGS. 6 and 7). The reference temperature 3 (formula symbol T_{B}) is determined by the reference temperature sensor 13. The parameters L, v, a, λ, and A are generally stored in the system as calibration data.

$$\dot{Q} = \frac{f ( \frac{uL}{v} , \frac{v}{a} ) λ}{L} ( {T}_{A} - {T}_{B} ) A \qquad ( 15 )$$

With known cross-sectional geometry 8 of the aorta 9 of the patient in the region of the support system (ascertainable, for example, by ultrasound, computer tomography, or magnetic resonance tomography), the total fluid volume flow 1 (formula symbol Q_{HTV}) can be determined from the flow speed u determined in this way. The corresponding relationship is specified in the following equation (16):

$${Q}_{HZV} = k ( u ) uO \qquad ( 16 )$$

Here, k(u) is a calibration factor dependent on the flow profile, u is the calculated flow speed, and O is the measured aortic cross section (cf. cross-sectional geometry 8).

The solution proposed here allows in particular one of the following advantages:
Fully implanted, in particular pump-integrated and/or automatic determination of Q_{HTV} instead of only Qₚ.
Anemometric measuring methods using the waste heat of a VAD motor instead of an additional heating element do not result in an additional heat input into the organism.
This also prevents additional current consumption, whereby the battery runtime of autonomous systems is extended.

1. A method for determining a total fluid volume flow of blood in a region of a cardiac support system, comprising:
determining a reference temperature of the blood,
determining a motor temperature of an electric motor of the cardiac support system,
determining a thermal dissipation loss of the electric motor, and
determining the total fluid volume flow based on the reference temperature, the motor temperature, and the thermal dissipation loss of the electric motor.

2. The method according to claim 1, further comprising heating the blood by the electric motor, wherein determining the reference temperature comprises measuring the reference temperature prior to heating the blood by the electric motor.

3. The method according to claim 1, wherein determining the motor temperature comprises measuring the motor temperature of the electric motor at a surface along which the blood flows.

4. The method according to claim 1, wherein determining the motor temperature comprises measuring the motor temperature of the electric motor inside the motor.

5. The method according to claim 1, further comprising determining a flow speed of the blood based on calibration data, the reference temperature, the motor temperature, and the thermal dissipation loss of the electric motor.

6. The method according to claim 1, wherein determining the total fluid volume flow is based in part on a cross-sectional geometry of an aorta in the region of the cardiac support system.

7. The method according to claim 1, further comprising determining a fluid volume flow of a portion of the blood flowing through the cardiac support system.

8. The method according to claim 1 wherein the reference temperature of the blood is a temperature of the blood in the region of the cardiac support system.

9. A computer readable storage medium storing therein computer-readable instructions that, when executed by a processing unit, cause the processing unit to:
determine a reference temperature of blood flowing in a region of a cardiac support system,
determine a motor temperature of an electric motor of the cardiac support system,
determine a thermal dissipation loss of the electric motor, and
determine a total fluid volume flow of the blood based on the reference temperature, the motor temperature, and the thermal dissipation loss of the electric motor.

10. The computer readable storage medium according to claim 9, wherein operation of the electric motor heats the blood and wherein the computer-readable instructions, when executed by the processing unit, are configured to cause the processing unit to determine the reference temperature by measuring the reference temperature prior to the electric motor heating the blood.

11. The computer readable storage medium according to claim 9, wherein the computer-readable instructions, when executed by the processing unit, are configured to cause the processing unit to determine the motor temperature by measuring the motor temperature of the electric motor at a surface along which the blood flows.

12. The computer readable storage medium according to claim 9, wherein determining the motor temperature comprises measuring the motor temperature of the electric motor inside the electric motor.

13. The computer readable storage medium according to claim 9, wherein the computer-readable instructions, when executed by the processing unit, are further configured to determine a flow speed of the blood based on calibration data, the reference temperature, the motor temperature, and the thermal dissipation loss of the electric motor.

14. The computer readable storage medium according to claim 9, wherein determining the total fluid volume flow is based in part on a cross-sectional geometry of an aorta in the region of the cardiac support system.

15. The computer readable storage medium according to claim 9, wherein the computer-readable instructions, when executed by the processing unit, are further configured to determine a fluid volume flow of a portion of the blood flowing through the cardiac support system.
